(19) United States
(12) Patent Application Publication
(10) Pub. No.: US 2012/0230967 A1
(43) Pub. Date: Sep. 13, 2012

(54) DEVICE AND METHOD TO ACTIVATE PLATELETS

(75) Inventors: Marc Long, Denville, NJ (US); Lin Song, Wayne, NJ (US)

(73) Assignee: HOWMEDICA OSTEONICS CORP., Mahwah, NJ (US)

(21) Appl. No.: 13/042,842

(22) Filed: Mar. 8, 2011

(51) Int. Cl. A61K 35/14; A61M 5/315; C12N 5/02; A61M 5/00; A61P 17/02; A61P 19/00

(52) U.S. Cl. 424/93.72; 435/375; 604/93.01; 604/218; 604/272

(57) ABSTRACT

The present invention includes a method for activating platelets in a platelet solution, the method may include passing the platelet solution through an activator at least once to activate at least one of the platelets in the platelet solution to produce an activated platelet solution. The activator may further include a flow disruption element. The method may further induce administering the activated platelet solution to the repair site of a patient. The present invention may also be a system for treating a medical condition at a repair site within a patient in need thereof, which may include a platelet solution container for housing a platelet solution; an activator to activate at least one platelet; and an introducer to direct the at least one activated platelet to the repair site.

### BACKGROUND OF THE INVENTION

Different techniques have been used in the medical arts to augment the body's natural regenerative processes. Among these techniques are cell-based therapies, which have been used with some success as hemostatic agents and to promote tissue repair and wound healing in vivo. For example, concentrated preparations of bone marrow cells comprising mesenchymal stem cells have been employed clinically as a means to facilitate bone repair and regeneration.

The use of platelets are of particular clinical interest, as these cells secrete a variety of cytokines that are critical to the healing process, including, for example: platelet-derived growth factor, platelet activating factor, transforming growth factor-beta, insulin like growth factor 1, epidermal growth factor, basic fibroblast growth factor, and vascular endothelial growth factor. These secreted factors also include chemotactic factors that can attract other healing factors to the site of tissue injury. Such components may collectively be referred to as “releasates.” Several platelet-based products designed to facilitate wound healing are commercially available and include several based on the use of autologous “platelet rich plasma” (PRP). See, e.g., Smith et al., J. Lancaster General Hospital, Vol. 2, No. 2, pp 73-78 (2007).

Typically, platelet-rich plasma solutions (PRP solutions) are injected into a repair site as is, after the centrifugation process is complete, and in an inactivated state. Occasionally, an activator, such as calcium chloride or calcium gluconate, is added to the PRP solution, just prior to injection, to initiate activation of a small fraction of the platelets in the PRP solution. These activators are usually added in liquid form to the PRP solution. Alternatively, such PRP solutions are added to a scaffold, such as a collagen scaffold, which may result in some activation of the platelets in the PRP solution.

When a PRP solution with inactivated platelets is added to a repair site, little if any assistance to the repair of the tissue site occurs. Similarly, if the PRP solution is first treated with thrombin, to initiate the activation, such activation is sudden and massive, leaving few releasates available at the repair site to assist in repair and healing. Alternatively, if calcium chloride is used, the concentration of the PRP solution platelet and releasate is diluted, due to the addition of the liquid calcium chloride. Moreover, the addition of such a liquid incorporates additional chemicals into the PRP solution, as well as the possibility of added unwanted materials, which are not necessary and could be adverse to the healing process. The addition of such chemicals such as exogenous biological substances may also introduce contaminations such as a virus, or trigger or instigate undesirable immune reactions in the clot recipient. Moreover, the addition of calcium chloride has been considered the culprit for additional undesirable post-surgical pain at the repair site.

### BRIEF SUMMARY OF THE INVENTION

In a first embodiment, the present invention includes a device for activating platelets in a platelet solution, which may be used to treat a medical condition at a repair site within a patient in need thereof, the device may include an actuator, the actuator including at least a columnar body having a length along a longitudinal axis and a flow disruption element positioned along at least a portion of the length. Additionally, the flow disruption element may be a plurality of projections positioned at an angle between and including about 0 degrees and about 90 degrees to the longitudinal axis. of a bore within the columnar activator, the plurality of projections are positioned along at least a portion of the length of the bore of the columnar activator. More specifically, the plurality of projections may be generally perpendicular to the longitudinal axis. Alternatively, the flow disruption element may be a helical spiral positioned within at least a portion of the length of the bore of the columnar activator. Alternatively, the flow disruption element may be a plurality of microspheres, nanospheres, or both positioned within at least a portion of the bore of the columnar activator. Alternatively, the flow disruption element may be a filter positioned within at least a portion of the bore of the columnar activator, on one end of the columnar activator, or both.

In another embodiment, the present invention includes a method for activating platelets in a platelet solution, the method may include passing the platelet solution through an activator at least once to activate at least one of the platelets in the platelet solution to produce an active platelet solution. The activator may further include a flow disruption element. The method may further include administering the activated platelet solution to a repair site of a patient. The platelet solution may be a PRP solution. The activated platlet solution may be administered through a syringe, a spray injector, or the like.

In yet another embodiment, the present invention may include a system for treating a medical condition at a repair site within a patient in need thereof, the system may include a platelet solution container for housing a platelet solution; an activator, through which the platelet solution passes to activate at least one of the platelets in the platelet solution; and an introducer to direct the at least one activated platelet to the repair site. Further, the introducer is a syringe, spray injector, cannulated liquid insertion device, or the like.

### BRIEF DESCRIPTION OF THE DRAWINGS

FIG. 1 illustrates a first embodiment activator of the present invention.

FIG. 2 illustrates another embodiment of an activator of the present invention.

FIG. 3a illustrates yet another embodiment of an activator of the present invention.

FIG. 3b illustrates a further embodiment of an activator of the present invention.

FIG. 4 illustrates yet another embodiment of an activator of the present invention.

FIG. 5 illustrates yet another embodiment activator of the present invention.

FIG. 6 illustrates one embodiment of a method of the present invention in which the repair site is within the knee joint.

FIG. 7 illustrates one embodiment of a system of the present invention including one embodiment of an activator of the present invention and related equipment.

FIG. 8 illustrates fold change of the presence of transforming growth factor beta (TGF-beta) in activated PRP solutions versus inactivated PRP solutions.

FIG. 9 illustrates fold change of the presence of platelet-derived growth factor beta (PDGF-B) in activated PRP solutions versus inactivated PRP solutions.

### DETAILED DESCRIPTION

The present invention includes a device, system, kit and method for activating platelets in a PRP solution using mechanical forces, such as shear, compression, and friction. Such activation does not incorporate any additional materials, chemicals, or elements to the PRP solution such as exogenous anti-coagulants or coagulation activators or the like. In one embodiment, the mechanical activation of the present invention provides for activation of a portion of the platelets in the PRP solution, resulting in a portion of the platelets releasing releasates. This may improve the efficacy of the PRP solution as the releasates (for example various growth factors and chemotactic agents) are released into the repair site immediately upon introduction, and may immediately affect the biological milieu. Additionally, extra releasate material may be added to the PRP solution in addition to those releasates contained in the platelets. The portion of the platelets that remain inactivated may then be available for future activation once at the repair site. The device, system, kit and method may be used on a PRP solution, a blood collection solution, a bone marrow solution, or any combination thereof, and may activate any of these solutions containing blood, bone marrow, or only platelets.

A PRP solution may be defined as any fluid, liquid, gel, or flowable material containing at least one platelet. The PRP solution may also include cells, cytokines, or any other elements suitable for application to a repair site within a patient in need thereof. The platelets, and other materials in the PRP solution, may be autologous, allogenic, syngeneic, xenogenic or synthetic.

For example, solutions which are enriched in platelets or stem cells may be produced by using blood or bone marrow as the biological sample, respectively, by methods known in the art. Then, as understood herein, these solutions, upon activation, may be used to treat any medical condition in which the local application of platelets or stem cells in vivo can achieve a beneficial effect. Such conditions are easily discerned by one of skill in the art and include, without limitation, medical conditions in which the site-specific delivery of platelet-derived cytokines, growth factors and other biological components produce a medical benefit in vivo, or in which implantation of a clot comprising progenitor stem cells may prove beneficial. For example, such clots may be used therapeutically to facilitate the healing of damaged or diseased tissue or joints, wounds, surgical incisions, or other various tissue injuries.

While the following devices, methods, systems and kits may be used on any of the above-referenced solutions to activate certain materials in those solutions, the PRP solution, in which at least one platelet is activated, will be used as the primary example throughout. A fraction or portion of the platelets in the PRP solution may be activated using the following devices, methods, systems and kits, or substantially all of the platelets in the PRP solution may be activated. Also, in one such example, any portion of the platelets may be activated prior to introduction of the PRP solution to the repair site, and the remainder of the platelets may be activated once at the repair site through natural or induced means.

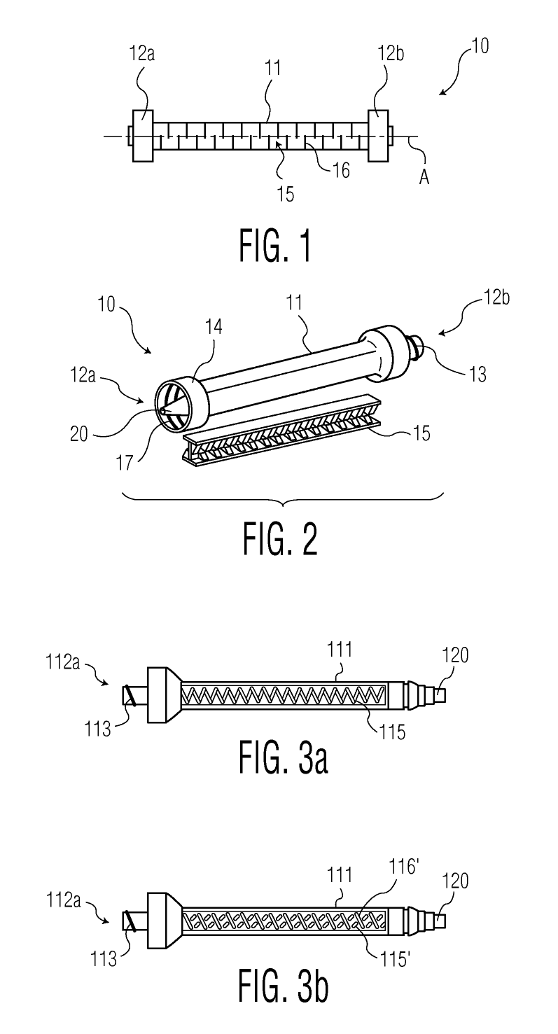

In a first embodiment, illustrated in FIGS. 1-2, the present invention may include an actuator 10, which may be sterile, for activating platelets in a platelet solution to treat a medical condition at a repair site within a patient in need thereof. The actuator 10 may include a columnar body 11 having a length along a longitudinal axis A and a flow disruption element 15 positioned along at least a portion of the length of the columnar body 11. The actuator 10 may further include a Luer Lok® (generically referred to as a luer lock connection) 12a, 12b on each end of the columnar body 11, which may be used to attach the actuator 10 to other structures such as a spray nozzle tip (not shown), second columnar body, syringe components (see below), or the like. One example of the basic structure of the columnar body and actuator may be a Series 180A Plastic Turbo (Square) Bell Mixer (Nordson EFD, East Providence, R.I., USA).

The columnar body 11 may be a generally cylindrical shape with a throughbore along the longitudinal axis A, within which the flow disruption element 15 may be positioned. The columnar body 11 may also be generally rectangular, generally elliptical, square, polygonal, or any other desired shape. The columnar body 11 itself may also take on a non-linear shape such as a curved column, a spiral column, or the like. The luer locks 12a, 12b may be positioned generally on either end of the columnar body 11 to provide attachment areas on either end of the throughbore of the columnar body 11. The luer locks 12a, 12b may be connected to typical blood and cell collecting devices and instruments, such as syringes, spray nozzles, needles, biopsy needles, blood collecting devices, instruments for collecting body fluid containing platelets, devices for storing, containing, delivering or handling a solution including platelets, or the like. The luer locks may also provide a sealed throughbore, representing the interior volume of columnar body 11, to maintain sterility of the interior volume of columnar body 11, and to provide a sealed volume in the event a flowable material must be stored, contained, transported, or the like, within the throughbore. Alternatively, the luer locks may not seal the interior and may instead only provide a connection point for additional elements. Luer locks 12a, 12b may be of any type known in the art, which would provide a universal connection site for any additional structures and instruments such as those above. Of course, alternatives to luer locks, such as standard valves, universal joints, and the like, may also be positioned on the columnar body 11.

FIGS. 1 and 2 illustrate one possible embodiment of the flow disruption element 15. The flow disruption element may include at least one projection 16, and alternatively a plurality of projections, extending into the volume of the columnar body. The projections 16 are intended to disrupt the flow of a platelet solution passing through the actuator 10. The flow disruption element 15 may be of any design and may be custom-made for various uses, for various levels of turbulence (which may affect the level of platelet activation), and for various types of turbulence. The projections may be generally perpendicular to the axis of the flow disruption element, as in FIG. 1, or the projections may be at an angle other than perpendicular to the axis, as in FIG. 2. FIG. 2 illustrates the projections at about a 45 degree angle to the axis, in a downstream direction, however, any angle, aimed upstream or downstream, is envisioned. For example, the projections may be positioned at an angle between and including about 0 degrees and about 90 degrees to axis A. The projections may be generally rectangular or may have a different shape such as triangular, or any other shape which may provide a stable projection capable of disrupting the path of the fluid therethrough.

FIG. 2 also illustrates one embodiment of luer locks 12a, 12b which may be included on the columnar body 11. One exemplary luer lock 12a may include a cylindrical outer portion 14 and an inner nozzle portion 20. The portion 14 may also have threads 17 on an inner surface, and may further either be stationary as to body 11, or alternatively may be rotatably connected to body 11. Luer lock 12b may include a portion having a narrowed diameter which may further include a thread 13 near the end surface. Using luer lock 12a and luer lock 12b as an example, if they were secured to two different structures, for example, a columnar body and a syringe needle, they are designed to be secured to one another wherein the nozzle 20 fits within the narrowed portion of luer lock 12b. Moreover, the threads 13 of luer lock 12b may be secured to the threads 17 of the inner surface on luer lock 12a. Thus, luer locks 12a and 12b may be universal, such that luer locks 12a, 12b may be on different devices such that the devices may be secured together. Of course, other attachment elements may be used other than this arrangement. Also, other securing devices may be used in place of the threads, such as a snap-fit structure, a taper fit, or the like.

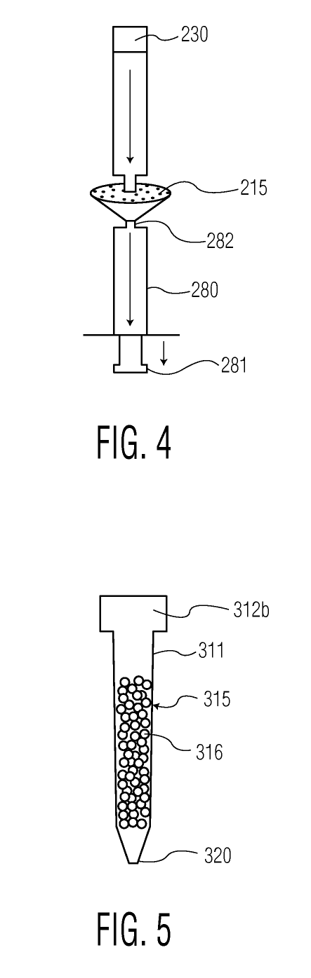

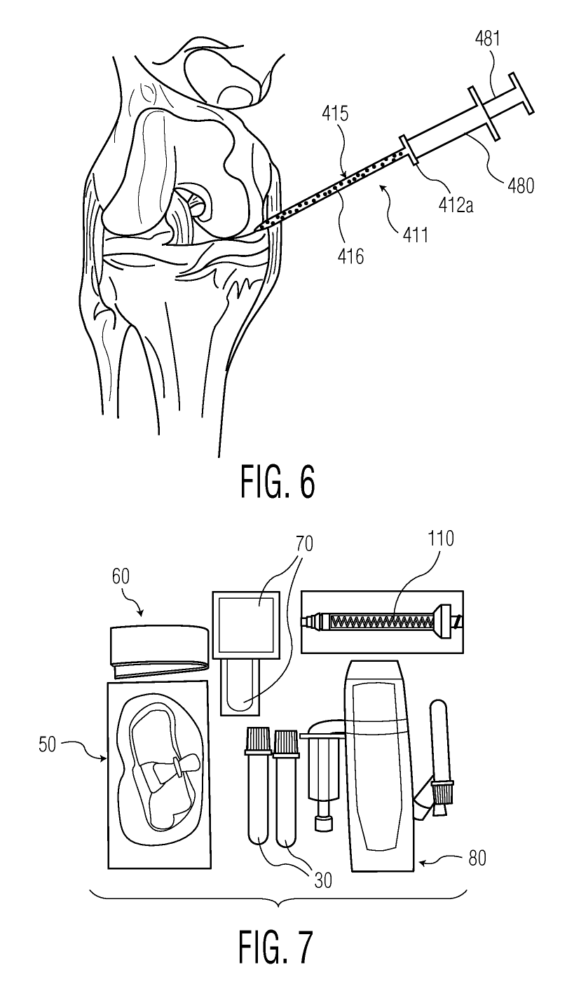

Alternative embodiments of flow disruption element (FIGS. 1-2) may include a helical spiral structure 115 (FIG. 3a) along at least a portion of the throughbore, a discontinuous spiral structure 115′ (FIG. 3b), a plurality of microspheres, nanospheres or both 315, 415 (FIGS. 5 and 6), or a filter 215 (FIG. 4). Other similar static flow disruption structures are also envisioned, and may be used within or in conjunction with the columnar body.

The helical spiral embodiment, illustrated in FIG. 3a, may include a flow disruption element 115 as a single helix, a double helix, multiple helixes, or any variation or combination of these. The helical spiral may impart a rotational motion to the flow of PRP solution through the columnar body 111. The helical spiral may also be “stepped,” by which the flow disruption element 115 includes alternating perpendicular and parallel portions, relative to the longitudinal axis A, while also continuing in a spiral fashion along at least a portion of the columnar body 111. FIG. 3a also illustrates a columnar body 111 including a nozzle portion 120 for expelling the PRP solution from the columnar body 111 to a receiving device or, alternatively, directly to a surgical site in a patient in need thereof. The other end of the columnar body 111 may include a luer lock 112a which may have a connection element, such as a thread 113, for connection to another luer lock on another device or structure, such as a plunger or other syringe device, PRP solution storage device, or the like.

FIG. 3b illustrates a second embodiment of a spiral flow disruption element 115′, in which the spiral shape may be discontinuous. In this embodiment, the discontinuous spiral shape may appear as a series of propeller-shaped structures 116′ stacked on top of one another. Each propeller-shaped structure 116′ may have at least one “blade” positioned circumferentially around an axis of the structure. For example, as illustrated, the structure may include two “blades,” each of which extend circumferentially about 180 degrees around the axis of the structure such that the two blades substantially cover the entire 360 degrees circumference around the axis. Of course, any number of blades may cover any portion of the 360 degrees around the axis, so long as the blades cover a sufficient circumference to disrupt the flow of a solution through the columnar body 111. Moreover, in an alternative embodiment each propeller-shaped structure has an opposite rotation to those other propeller-shaped structures adjacent to it. Thus, a first structure may have a clockwise rotation, a second structure may have a counterclockwise rotation, a third structure may have a clockwise rotation, and so on. Additionally, each propeller-shaped structure may be is rotated about 90 degrees relative to the structures adjacent to it. Thus, these variations may create an even greater disruption of flow of solution through the columnar body 111 than a typical spiral structure (FIG. 3a) because the fluid must change course as it flows past each successive propeller-shaped structure. The propeller-shaped structures of FIG. 3b are stationary relative to one another and the columnar body 111, though it is envisioned that the structures may rotate about their axes relative to the columnar body 111, as a group, or relative to one another individually.

In yet another embodiment, as illustrated in FIG. 4, the flow disruption element 215 may be a filter, or a series of filters, having porosity or porosities sufficient to disrupt the flow of a platelet solution therethrough to activate at least one platelet. The filter or filters may be positioned between a platelet solution holding container 230 and a syringe 280, which may have a syringe tip 282 and a plunger 281, for use in introducing the filtered platelet solution to a repair site. Alternatively, the filter or filters may be positioned within a columnar body of an activator, similar to the other embodiments discussed above.

In an alternative embodiment, as illustrated in FIGS. 5 and 6, the columnar body 311, 411 may house a flow disruption element 315, 415, which may include packed particulates, such as a plurality of microspheres, nanospheres or both 316, 416—the difference being the size of the specific spheres, one having a micron scale, the other a nano scale, respectively. The plurality of microspheres, nanospheres, or both are packed within at least a portion of the throughbore of the columnar body 311, 411 to disrupt, constrict and shear the flow of platelet solution therethrough. The particulates may be porous or solid, such that porous particulates may allow the platelets to pass through the particulates, in addition to passing around the particulates, and be of any material suitable and compatible for use with blood and other bodily fluids.

Further, any combination of the above columnar body shapes and flow disruption element may be used together. Moreover, the columnar body and flow disruption elements may include multiple shapes or structures along the length of the activator to provide various types of mechanical forces on the PRP solution. Additionally, any other type of static mixer or flow disruption elements may be used that imparts constriction, turbulence or other mechanical forces on a PRP solution sufficient to activate at least one platelet via such mechanical forces.

In use, the various embodiments of the activator 10, discussed above, activate at least one platelet in a platelet solution to create an activated platelet solution, which may be used for application to a repair site within a patient in need thereof. Exemplary in vivo repair sites include a joint (see FIG. 6), a soft tissue attachment site, a soft tissue having low vascularization, a soft tissue which suffered a trauma or injury, or the like.

The at least one platelet, fraction or portion of platelets, or substantially all of the platelets, may be activated upon passing the PRP solution through the activator because the activator creates a tortuous pathway along which the PRP solution must flow. The tortuous pathway may create turbulence in the PRP solution flow, which imparts sufficient mechanical stress to the PRP solution to activate at least one platelet in the PRP solution and thus forming the activated platelet solution. As discussed further below, the PRP solution may be passed through the activator at any point prior to delivery to the surgical site of a patient. For example, the PRP solution may be passed through the activator as the solution is being drawn from a storage container. In this example, the activator may be positioned, using its luer locks, between a plunger structure and a needle, such that as the plunger is drawn backwards, to pull PRP solution through the needle from the storage container, the PRP solution must pass through the activator, thus activating at least one platelet. Alternatively, a typical syringe may be used to collect the PRP solution which is then passed through an activator prior to implantation. In a further example, the activator may be part of a delivery device, which again may include a plunger and a syringe needle, such that as the plunger is pressed to deliver the PRP solution to the surgical site, it must pass through the activator prior to passing through the needle and on to the surgical site (as in FIG. 6).

The tortuous pathway may cause the PRP solution to constrict, shear, invert, rotate, mix, become intermittent, or the like, which causes at least one platelet in the solution to activate. The use of mechanical or physical forces may eliminate the need for biochemical activators, as discussed above, to activate the platelets in the PRP solution for, for example orthopedic uses at a repair site.

Furthermore, in some embodiments, a pressure differential may be required to cause activation of the at least one platelet as the force caused by gravity alone may be insufficient to cause activation, i.e., rupture of a platelet. Thus, an increased pressure, such as a pressure greater than 1 atmosphere, may be used to force the PRP solution through the activator to increase the forces of shear and constriction. In one embodiment, a plunger may be secured to the activator such that pulling or pressing of the plunger will create an increased pressure on the PRP solution to force the solution through the activator in one direction.

The activator embodiments of the present invention may be made from any material suitable for use with blood and other bodily fluids, and particularly for blood or other bodily fluids, that will be injected into a patient. For example, polypropylene or other such plastics may be used. Alternatively, the activator may be constructed of a material which may, upon contact with the PRP, initiate activation of the platelets. Such materials may not require a convoluted pathway or other flow disruption element. Such materials include glass tubes, plasma-treated polymeric tubes, glass-coated tubes, silica coated tubes, or containers otherwise internally coated with silica particles or a glass or silica layer. The activator may be constructed from one of these materials and may also include a flow disruption element, constructed of either one of these materials or of a different material, such that activation occurs upon platelets contacting the surface of the activator (and flow disruption element if made of one of these silica materials as well) and upon mechanical mixing caused by the flow disruption element. Using a combination of a silica material (for the columnar body) and a flow disruption element, which may also be made of a silica material or other material, may increase the number of platelets activated in the PRP above the number which would be activated if the flow disruption element or silica material were present on its own.

The devices, methods, systems and kits of the present invention may produce partial or complete activation of an at least one platelet. The activation state of the at least one platelet may be temporary and reversible, or terminal. As an example, an activated platelet may be activated to a level that the shape of the platelet is changed, but no releasates, such as alpha-particles and growth factors, are released from the platelet, and no clotting is obtained.

Activation of platelets forces the platelets to initiate the natural clotting sequence through release of the releasates (cytokines, growth factors, fibrin, etc.). The releasates may be useful in enhancing the biological environment where the activated platelets are being used, and in producing a chemotactic milieu, such as accelerating or enabling healing and tissue repair, influencing the onset or progression of a disease at a repair site, or the like.

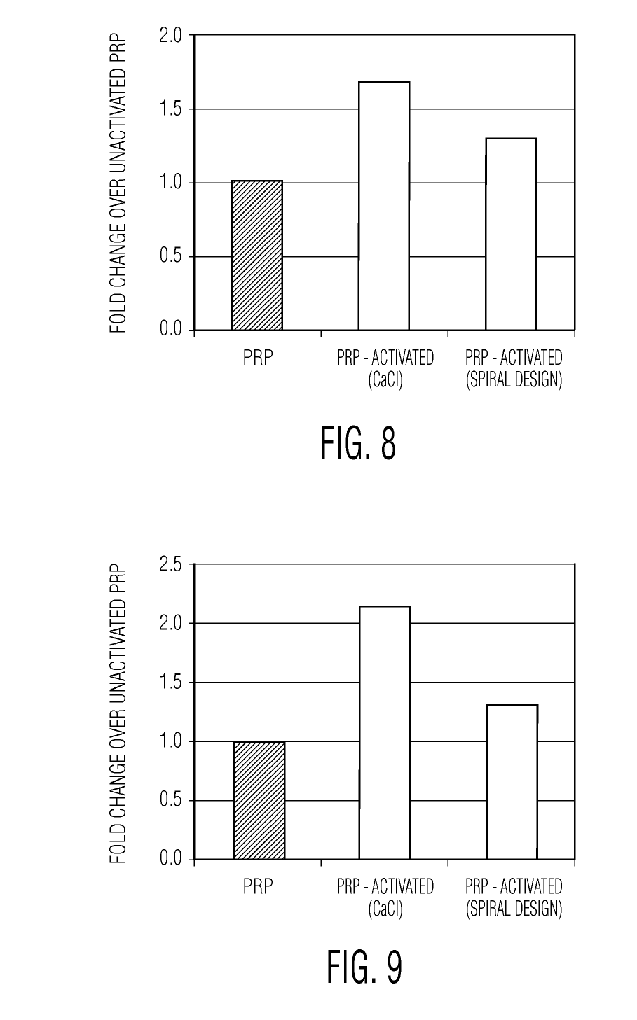

FIGS. 8 and 9 illustrate data as to the level of activation of a PRP solution based on swine blood, using two blood samples collected from a castrated male Yorkshire pig. The blood samples were centrifuged using a PCO2 centrifuge (Societe PROCESS Sarl, Nice, France) for 6 minutes at 3700 RPM and another 6 minutes at 4500 RPM. The static mixer was manufactured by Nordson EFD and included a discontinuous spiral design (FIG. 3b) specifically designed based on the disclosure of this invention (manufactured to specification by Plastic Spiral (Helix) Bell Mixer). A portion of each sample was activated with calcium chloride, and another portion of each sample was passed through the static mixer. The left-hand bar is the inactivated PRP solution, and the middle bar is the control in which the PRP solution is chemically activated with calcium chloride. The right-hand bar illustrates the level of activation of platelets in a PRP solution using the embodiment of FIG. 3b, namely the activator having a discontinuous spiral flow disruption element 115′. FIG. 8 illustrates the fold increase in the presence of TGF-beta, which is directly proportional to the fold increase in activated platelets in the PRP solution. Likewise, FIG. 9 illustrates the fold increase in the presence of PDGF-B, which is also directly proportional to the fold increase in activated platelets in the PRP solution.

FIGS. 8 and 9 show that the spiral activator (right-hand bar), using mechanical forces, does increase growth factor concentration, and therefore induces at least a portion of the platelets in the PRP solution to activate. Additionally, since the increase in activation is less dramatic than when chemical activation is used, the surgeon may better control the amount of activation by passing the platelets through the activator a specific number of times to obtain the desired level of activation.

In another embodiment, a method for activating platelets in a platlet solution, such as a PRP solution, which may be used to treat a medical condition at a repair site within a patient in need thereof, may include preparing a PRP solution from a blood source, passing the PRP solution through an activator at least once to activate at least one platelet in the PRP solution to produce an activated platelet solution, and introducing the activated PRP solution to the repair site. As above, this method may also be used with a solution derived from a bone marrow source. The passage of the PRP solution through the activator may occur as the solution is drawn from the blood source or storage source, or alternatively upon introduction of the PRP solution to the repair site, or both. The step of passing the solution through the activator may also be an intermediate step between collecting the PRP solution and delivering it to the repair site, or again, passage through the activator may occur at any two or even all three steps (collection, intermediate, and introduction).

In yet another embodiment, a method is provided for activating platelets in a platlet solution, such as a PRP solution, which may be used to treat a medical condition at a repair site within a patient in need thereof, which may include passing the platelet solution through an activator at least once to activate at least one of the platelets in the PRP solution, to produce an activated PRP solution. The activator may further include a flow disruption element. The method may further include administering the activated platelet PRP solution to the repair site of the patient. If the PRP solution is passed through the activator additional times, a greater amount of platelets in the PRP solution may be activated. In one example of this method, the PRP solution is prepared using a centrifuged blood sample, and is aspirated into the activator using a blood transfer device (such as a tube or the like). The activator is connected to a syringe using a luer lock or like structure, which may capture the PRP solution upon exiting the activator. At least one platelet in the PRP solution may be activated upon exiting the activator, and as such, once the activated PRP solution passes through the activator and into the syringe, the PRP solution is ready for use. As above, a plunger on the syringe may be used to force the solution through the activator to create a pressure differential which may assist in activation of the platelets. The syringe may then be disconnected from the activator and moved adjacent to the repair site to introduce the at least one activated platelet PRP solution to the repair site.

FIG. 6 illustrates one example of a method where the repair site is located in the knee joint (the patella has been reflected for ease of illustration). In this embodiment, the activator is part of a syringe, which includes a syringe body 480 and a plunger 481, the syringe is connected to luer lock 412a of columnar body 411. The flow disruption element 415 includes particulates, but any flow disruption element may be used. In this example, the PRP solution may be formed from a blood sample using centrifugation, and the resulting PRP solution may be aspirated via a blood transfer device (such as a tube or the like) into the syringe 480, which is attached via luer lock 412a to the flow disruption element 415. Once the repair site, e.g., meniscus, cartilage, tendon or ligament connection site, etc., is accessed through known means, a distal tip of the columnar body 411 is brought adjacent to the repair site. Alternatively, a needle may be attached to the distal tip of the columnar body, via a luer lock or other connection, and the distal tip of the needle may be positioned at the intended repair site. Or, the distal tip may include a spray nozzle. The plunger 481 is depressed, forcing the PRP solution through luer lock 412a and into flow disruption element 415. As the PRP solution passes through the flow disruption element 415, at least one platelet in the PRP solution is activated, and as such releasates are released from the at least one activated platelet, to form an activated PRP solution. The now activated PRP solution continues through the flow disruption element 415 until it exits from the distal tip of the columnar body 411 and is introduced into the repair site. The inactivated platelets and releasates from the activated platelets then initiate the clotting and healing process, as needed, at the repair site, which may include further activation of the inactivated platelets. This method may be performed in conjunction with other orthopedic surgical methods, such as, for example as to the knee joint, microfracture, meniscus tear repair, ligament or tendon replacement or repair, joint replacements, or the like. Alternatively, this method may be used on its own, and further, if a needle is used on the distal end of the activator, may be performed percutaneously and without the need for formal surgery. The activated platelet solution may be introduced anywhere into the joint as needed, including, but not limited to, the outer surface of tissue, injected into tissue, placed between tissues or tissue layers, or the like.

In a further embodiment, the present invention may include a kit for activating platelets in a platelet solution to treat a medical condition at a repair site within a patient in need thereof, some elements of which are illustrated in FIG. 7, which may include at least one blood separation unit (may be other tubes), at least one activator 110, a blood transfer device 80 (from the units 30 to the syringe or activator), at least one syringe, at least one needle, and a blood collection device 50. The kit may further include a tourniquet 60, gauze, wraps and bandages 70, and the like. Generally, the kit may include elements and devices for use in any or all of the steps for performance of the above methods including, for example, collection of a blood or other fluid from a source, separation and concentration processes (e.g., to form a PRP solution), activation of the solution, and delivery to a repair site.

The kit may include certain elements, and may exclude others, or may include multiples of certain elements, depending on the intended repair site or sites for use of the kit. Further, any combination of columnar bodies and flow disruption elements may be included in the kit, which may be chosen based on user preference or based on the intended repair site. The elements of the kit may be sterilized and disposable. Additionally, while the above elements of the kit may be disposable, the kit may also include a centrifuge for use in preparation of the PRP solution. For example, a user, such as a doctor or a hospital, may order a first kit which includes the centrifuge, but subsequent kits ordered by the doctor or hospital may not include the centrifuge as that device may be reused with the subsequent kits. Moreover, the activator may be sold separately from the rest of the kit, such that the basic solution preparation kit can be sold as is, and then the preferred activator can be ordered separately.

In a further embodiment, the present invention may include a system for treating a medical condition at a repair site within a patient in need thereof, the system may include a platelet solution container for housing a platelet solution; an activator, through which the platelet solution passes to activate at least one of the platelets in the platelet solution; and an introducer to direct the at least one activated platelet to the repair site. The platelet solution may be a PRP solution, and the container housing the PRP solution may be an at least one evacuated tube or container. The activator included in this system may be any of the activators discussed above, or may include a plurality of activators of any combination, as disclosed above. Further, the introducer maybe a syringe, spray injector, cannulated liquid insertion device, or the like. The system may further include a centrifuge for separating and concentrating a blood sample to form the PRP solution.

In yet a further embodiment, the present invention may include a system for treating a medical condition at a repair site within a patient in need thereof, which may include an activator and a syringe. The system may further include any of a centrifuge, which may be used to prepare the PRP solution, at least one blood separation unit 30 such as an evacuated tube, a blood transfer device 80 (from the evacuated tube to the syringe or activator), at least one needle, a blood collection device 50, a tourniquet 60, gauze, wraps and bandages 70, or the like. The system may be sufficient to perform each step of the above methods, including obtaining a blood sample, preparing the PRP solution, activating the platelets in the PRP solution to form an activated PRP solution, and introducing the activated platelet solution into the repair site of a patient. Additionally, for example, the system may include multiple activators such that a user of the system can perform multiple passes of the PRP solution through the activator to obtain the desired level of activation.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for activating platelets in a platelet solution, comprising:
passing the platelet solution through an activator at least once to activate at least one of the platelets in the platelet solution to produce an activated platelet solution.

2. The method of claim 1, wherein the activator comprises a flow disruption element.

3. The method of claim 1, further comprising administering the activated platelet solution to a repair site of a patient.

4. The method of claim 3, wherein the activated platelet solution is administered to the repair site through a syringe.

5. The method of claim 3, wherein the repair site is selected from the group consisting of a joint, a soft tissue attachment site, a soft tissue having low vascularization, and a soft tissue which suffered a trauma or injury.

6. The method of claim 2, wherein the activator further comprises a columnar shape having a bore therethrough, and the flow disruption element is positioned within the bore of the columnar shape.

7. The method of claim 6, wherein the flow disruption element comprises at least one projection positioned at an angle between and including about 0 degrees and about 90 degrees to a longitudinal axis of the bore within the columnar activator, the at least one projection is positioned along at least a portion of the length of the bore of the columnar activator.

8. The method of claim 6, wherein the flow disruption element comprises a helical spiral positioned within at least a portion of the length of the bore of the columnar activator, wherein the helical spiral may extend along substantially the entire length of the columnar activator or may be discontinuous in at least one position along the length of the columnar body.

9. The method of claim 6, wherein the flow disruption element comprises a plurality of microspheres or nanospheres positioned within at least a portion of the bore of the columnar activator.

10. The method of claim 6, wherein the flow disruption element comprises a filter positioned within at least a portion of the bore of the columnar activator, on one end of the columnar activator, or both.

11. The method of claim 1, wherein the activator is positioned within a syringe, such that the platelet solution passes, by depressing a plunger, through the activator to activate the at least one platelet, and further depression of the plunger passes the at least one activated platelet from the activator to a syringe tip, for introducing the activated platelet solution to a repair site.

12. The method of claim 1, wherein substantially all of the platelets in the platelet solution are activated by the activator.

13. The method of claim 2, wherein the platelets are activated by mechanical forces asserted on the platelet solution by the flow disruption element.

14. The method of claim 1, wherein the activated platelets are immediately introduced to a repair site of a patient upon activation.

15. The method of claim 1, wherein the at least one activated platelet is not activated by a biochemical activator.

16. The method of claim 1, wherein the platelet solution is a platelet-rich plasma solution obtained by centrifugation of a whole blood sample.

17. A system for treating a medical condition at a repair site within a patient in need thereof, comprising:
a platelet solution container for housing a platelet solution;
an activator, through which the platelet solution passes to activate at least one of the platelets in the platelet solution; and
an introducer to direct the at least one activated platelet to the repair site.

18. The system of claim 17, wherein the introducer is a syringe, spray injector, or cannulated liquid insertion device.

19. The system of claim 17, wherein the introducer comprises a syringe, and the activator is positioned within the syringe, such that a plunger of the syringe is positioned on one end of the activator and a syringe tip is positioned on an other end of the activator.

20. The system of claim 17, wherein the activator further comprises a flow disruption element selected from the group consisting of a plurality of projections at an angle between and including about 0 degrees and about 90 degrees to a longitudinal axis of the activator, a helical spiral, a discontinuous helical spiral, a plurality of microspheres, a plurality of nanospheres, a plurality of both microspheres and nanospheres and a filter.
